(19) United States
(12) Patent Application Publication
(10) Pub. No.: US 2009/0238785 A1
(43) Pub. Date: Sep. 24, 2009

(54) Novel Aldehydic Musks and Derivatives Thereof

(75) Inventor: Luca Turin, Somerville, MA (US)

Correspondence Address: WOMBLE CARLYLE SANDRIDGE & RICE, PLLC, ATTN: PATENT DOCKETING, P.O. BOX 7037, ATLANTA, GA, 30357-0037, US

(73) Assignee: Flexitral, Inc., Chantille, VA (US)

(21) Appl. No.: 11/577,776

(22) PCT Filed: Jun. 24, 2005
(86) PCT No.: PCT/US05/22279

§ 371 Date: May 20, 2009

Related U.S. Application Data
(60) Provisional application No. 60/582,219, filed on Jun. 24, 2004.

(51) Int. Cl. A61K 8/33; A61L 9/01; A23G 4/00; A23L 1/22; A61K 31/11; C07C 47/42

(52) U.S. Cl. 424/65; 424/76.1; 426/3; 426/534; 514/699; 514/700; 568/425; 568/440; 512/17; 510/105; 252/186.1

(57) ABSTRACT

A compound comprising a Schiffs base of an aldehydic musk aromachemical with a substantially nonvolatile, odorless amine, wherein the imine moiety of said Schiffs base compound is stable against oxidation to a carboxyl group or a mixture of said compounds; said Schiffs base compound being biodegradable over time to said aldehydic musk and said nonvolatile, odorless amine; as well as compositions, products, preparations or articles having improved aroma, fragrance or odor characteristics containing as active ingredient such compound or mixture of compounds; the aldehydic musks themselves, and methods for the preparation of the Schiff bases and methods of imparting fragrance characteristics to substrates.

### FIELD OF THE INVENTION

The invention relates to aldehyde musk aromachemicals, fragrance and flavor compounds and derivatives thereof.

### DESCRIPTION OF THE PRIOR ART

Most aromachemicals or fragrance compounds with a musk-like aroma characteristic in general use today are ketones, typically methylketones. The so-called “aldehyde musks”; i.e., aromatic or polycyclic musks containing an aldehydic rather than a ketonic functional group, often far more potent than their ketonic cousins, are inherently unstable at the aldehyde moiety to oxidation to the corresponding carboxylic acid, thereby losing their fragrance characteristics. As a result of this inherent oxidative instability, the aldehydic musks are generally only of historical and academic interest and have fallen into disfavor and relative disuse. A typical such aldehydic musk is 2,4-di-tertiarybutyl-5-methoxybenzaldehyde, described in U.S. Pat. No. 2,450,879 and briefly marketed some 50 years ago as Ambral before being superseded by the more stable ketonic musks.

However, the ketone musks themselves have recently fallen into disfavor due to their relative stability in the environment into which they are released when employed in the industry. Recently there has been an outcry for the banning of the ketone musks because of their deleterious effect on the environment. Thus, recently, the European Council has recently stringently regulated the use of ketone musks—see XXIX Commission Directive 2003/16/EC of 19 Feb. 2003 adapting to technical progress Annex III to Council Directive 76/768/EEC.

The WWF has recently called for the banning of musk ketones—see http://www.ngo.grida.no/wwfneap/Publication/briefings/Musk.pdf. It is further disclosed therein that the European Scientific Committee on Cosmetics has concluded that human exposure to musk xylene and musk ketone should be reduced.

In [http://www.fpinva.org/Summary/environmental_concerns.htm] it is disclosed that the levels of musk ketones should be greatly reduced because of their effect on the environment. See also http://www.thenose.ch/acrobats/Musks_in_Perfumery.pdf; http://www.separationsnow.com/basehtml/SepH/1,0-5-7-0-43457-ezine-0-3,00.html; http://www.ospar.org/documents/dbase/publications/p00200_BD%20on%20musk%20xyle ne.pdf; http://www.ncbi.nlm.nih.gov/entrez/query.fcgi?cmd=Retrieve&db=PubMed&list_uids=11460697&dopt=Abstract;

It is an object of the invention to provide novel derivatives of aldehydic musks that are stable to oxidation to the carboxylic acid at the aldehyde moiety, that are biodegradable to environmentally safe products and that are capable of releasing the odorant aldehydic musk over time and as desired.

### SUMMARY OF THE INVENTION

The above and other objects are realized by the present invention, one embodiment of which relates to a compound comprising a Schiff's base of an aldehydic musk aromachemical with a substantially nonvolatile, odorless amine, wherein the imine moiety of the Schiff's base compound is stable against oxidation to a carboxyl group or a mixture of the compounds; the aldehydic musk aromachemical having up to 18 carbon atoms; the Schiff's base compound being biodegradable over time to the aldehydic musk and the nonvolatile, odorless amine.

Another embodiment of the invention concerns a method of stabilizing aldehydic musks against oxidation of their aldehydic groups to carboxyl groups, while preserving their odor, fragrance and flavor characteristics, comprising forming Schiff's bases thereof with substantially nonvolatile amines.

An additional embodiment of the invention comprises a composition, product, preparation or article having improved aroma, fragrance or odor releasing characteristics containing as active ingredient a compound or mixture of compounds described above. Still further embodiments of the invention relate to methods of conferring, improving, enhancing or modifying the taste, flavor or odor properties of a composition, product or article by treatment with one or more of the above-described Schiff's bases. Additional embodiments concern novel aldehydic musks.

### DETAILED DESCRIPTION OF THE INVENTION

As noted above, the use of aldehydic musks has fallen into disfavor, in spite of their superior potency to the ketonic musks, because of the inherent instability of the aldehyde group thereof to oxidation to the corresponding carboxylic acid. Thus, there exists a need in the art for methods and compositions for making such highly potent aldehydic musks available for efficient use.

The present invention is predicated on the discovery that the inherent oxidative instability of the aldehydic musks may be may be cured by derivatizing the aldehyde group thereof with amines to form Schiff's bases, i.e., imines. The use of certain amines for this purpose not only stabilizes the musks against oxidation but also gives rise to compositions wherein the fragrance characteristics of the aldehydic musks are preserved for certain applications. The Schiff's bases of the present invention slowly biodegrade over time to release the fragrant aldehydic musk and a non-volatile amine that imparts substantially no aroma or flavor of its own to the environment.

For certain applications such as, for example, laundry detergents, it is known to covalently bond fragrance molecules to amine group-containing polymers that are substantive to fabrics. Upon washing the fabric with the detergent, the polymer attaches to the fabric and survives the rinse and dry cycles. The polymer-fragrance Schiff's base slowly degrades over time to release the odorant, thereby providing the fabric with a long-lasting pleasant fragrance. Although it is stated in U.S. Pat. Nos. such as 6,699,823; 6,511,948; 6,413,920 and 6,566,312 that this expedient may be applied to “aldehydes” as well as ketones, there has been no disclosure in the prior art that would enable one skilled in the art to derivatize an aldehydic musk to form a Schiff's base that 1) stabilizes the aldehyde group thereof against oxidative decomposition to the carboxyl group, 2) is itself non-fragrant and 3) biodegrades over time to produce the fragrant aldehydic musk and a non-fragrant amine.

While not wishing to be bound by any theory of the mechanism of the invention, it is thought that the perfume components are released upon breaking down of the imine bond, leading to the release of the aldehydic musk and of the primary amine compound. This is believed to be achieved by either hydrolysis, photochemical cleavage, oxidative cleavage, or enzymatic cleavage. Still other means of release for the fragrance compound include elevated temperatures, e.g., the step of ironing a fabric treated with the Schiff base, tumble-drying, and/or wearing the fabric supplies the required heat to break down the Schiff's base.

The compositions of the invention may be utilized in and for applications where there is a need for a delayed release of the aldehydic musk. This includes compositions for use in rinses such as softening compositions, personal cleansing compositions such as shower gels, deodorants, bars, shampoos; stand alone compositions such deodorizing compositions, insecticides, and the like.

One preferred application concerns those involving contacting the compound of the invention with fabric. The compositions of the invention are suitable for use in any step, for example, of a domestic laundering operation, such as pre- and/or post-treatment compositions, as wash additives, as a composition suitable for use in the rinse process. Obviously, multiple applications can be made such as treating the fabric with a pre-treatment composition of the invention and thereafter with the composition suitable for use in the rinse process and/or drying process. By compositions suitable for use in the rinse process, these are to be understood to include compositions such as rinse added fabric softener compositions and dryer added compositions (e.g., sheets) which provide softening and/or antistatic benefits, as well as rinse additives.

Suitable aldehydic musks for derivitization according to the present invention include the aromatic (benzenic), and polycyclic musks.

The benzenic musks suitable for the present invention are 2,4-di-tertiarybutyl-5-methoxybenzaldehyde as well as those having the formula:

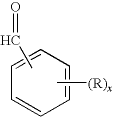

wherein: R is a straight or branched chain, saturated or unsaturated hydrocarbyl group; preferably, alkyl or alkenyl, having 1-8 carbon atoms, and
x is 1-5

An exemplary benzenic aldehydic musk is 2,4-ditertiarybutyl-5-methoxybenzaldehyde, having the structure:

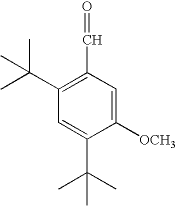

Suitable polycyclic musks are those having the structures:

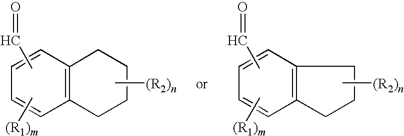

wherein: R₁ and R₂ may be the same or different and are R is a straight or branched chain, saturated or unsaturated hydrocarbyl group; preferably, alkyl or alkenyl, having 1-8 carbon atoms, and
m is 1-4 and n is 1-6.
   Typical such polycyclic musks are those having the structures:

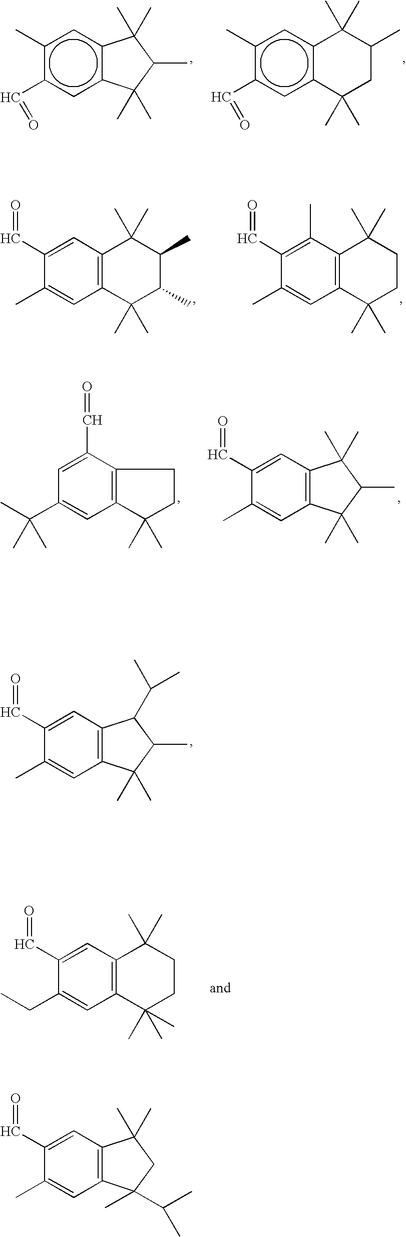

Suitable amines for forming the Schiff's bases with the aldehydic musks are preferably non-fragrant, odorless, non-volatile amines having a relatively low vapor pressure and higher molecular weight, i.e., aromatic or aliphatic amines containing more than about 12 carbon atoms. It has been suggested to form Schiff bases of aldehydic fragrance compounds or aromachemicals; however, in all such cases volatile amines such as alkyl anthranilates have been utilized that impart their own, sometimes objectionable, odor to the environment when the Schiff base degrades, thereby adversely impacting on the fragrance characteristics of the aromachemical. Suitable amines for use in the present invention include, for example, odorless, low vapor pressure aliphatic or aromatic amines containing at least one free, unmodified primary and/or secondary amino group such as, e.g., aminobenzenesulfonic acid, anthranilic acid, farnesyl amine, amino acids, such that the reaction of the amino end with the aldehyde will still leave a free carboxylic acid, thereby rendering the product Schiff base nonvolatile and odorless. In general all amino group containing zwitterions will be effective. By “primary and/or secondary amine”, it is meant a component which carries at least one primary and/or secondary amine and/or amide function.

In addition, amino functional polymers containing at least one amine group may be used in the practice of the invention. The general structure for amino functional polymers containing at least one primary amine group of the present invention, is as follows:

(NH2)n—[B]

wherein n is an index of at least 1 and B is the polymer backbone. B can optionally comprise a branching group. Amino functional polymers usable in the practice of the invention include those containing a secondary amine group have a structure similar to the above excepted that the polymer comprises one or more —NH— groups instead of —NH2 groups. Further, the polymer structure may also have one or more of both —NH2 and —NH— groups.

The amino functional polymer of the present invention contains at least one free, unmodified primary and/or secondary amino group attached to the main chain by hydrogen substitution, or by other suitable insertion. Also suitable is the amino functional polymer comprising an unmodified primary and/or secondary amino group present on side chain(s).

Preferably, the amino functional polymers of the present invention will comprise more than one amino groups, more preferably more than 10 amino groups. The amino functional polymers of the present invention will preferably present a molecular weight (MW) ranging from 400-50,000; most preferably from 600 to 40,000.

The amino functional polymer can be a linear homo-, co-polymer and optionally branched, grafted and/or cross-linked.

Preferred examples of suitable amino-functional polymers for use in the present invention are selected from the polyvinylamines, derivatives thereof, copolymer thereof, alkylene polyamine, polyaminoacids and copolymer thereof, cross-linked polyaminoacids, amino substituted polyvinylalcohol, polyoxyethylene bis amine or bis aminoalkyl, aminoalkyl piperazine and derivatives, N,N′-bis-(3-aminopropyl)-1,3-propanediamine linear or branched (TPTA), and mixtures thereof.

Polyamino acid is one suitable and preferred class of amino-functional polymer. Polyaminoacids are compounds which are made up of amino acids or chemically modified amino acids. They can contain alanine, serine, aspartic acid, arginine, valine, threonine, glutamic acid, leucine, cysteine, histidine, lysine, isoleucine, tyrosine, asparagine, methionine, proline, tryptophan, phenylalanine, glutamine, glycine or mixtures thereof. In chemically modified amino acids, the amine or acidic function of the amino acid has reacted with a chemical reagent. This is often done to protect these chemical amine and acid functions of the amino acid in a subsequent reaction or to give special properties to the amino acids, like improved solubility. Examples of such chemical modifications are benzyloxycarbonyl, aminobutyric acid, butyl ester, and pyroglutamic acid. More examples of common modifications of amino acids and small amino acid fragments can be found in the Bachem, 1996, Peptides and Biochemicals Catalog.

Preferred polyamino acids are polylysines, polyarginine, polyglutamine, polyasparagine, polyhistidine, polytryptophane or mixtures thereof. Most preferred are polylysines or polyamino acids where more than 50% of the amino acids are lysine, since the primary amine function in the side chain of the lysine is the most reactive amine of all amino acids. The preferred polyamino acid has a molecular weight of 500 to 10,000,000, more preferably between 5,000 and 750,000.

The polyamino acid can be cross linked. The cross linking can be obtained for example by condensation of the amine group in the side chain of the amino acid like lysine with the carboxyl function on the amino acid or with protein cross linkers like PEG derivatives. The cross linked polyamino acids still need to have free primary and/or secondary amino groups left for reaction with the active ingredient. The preferred cross linked polyamino acid has a molecular weight of 20,000 to 10,000,000, more preferably between 200,000 and 2,000,000.

The polyamino acid or the amino acid can be co-polymerized with other reagents like for instance with acids, amides, acyl chlorides. More specifically with aminocaproic acid, adipic acid, ethylhexanoic acid, caprolactam or mixture thereof. The molar ratio used in these copolymers ranges from 1:1 (reagent/amino acid (lysine)) to 1:20, more preferably from 1:1 to 1:10. The polyamino acid like polylysine can be partially ethoxylated.

Examples and supply of polyaminoacids containing lysine, arginine, glutamime, asparagine are given in the Bachem 1996, Peptides and Biochemicals catalog.

The polyaminoacid can be obtained before reaction with the active ingredient, under a salt form. For example polylysine can be supplied as polylysine hydrobromide. Polylysine hydrobromide is commercially available from Sigma, Applichem, Bachem and Fluka. Examples of suitable amino functional polymers containing at least one primary and/or secondary amine group for the purpose of the present invention are:
Polyvinylamine with a MW of about 300-2.10E6;
Polyvinylamine alkoxylated with a MW of about 600, 1200 or 3000 and an ethoxylation degree of 0.5;
Polyvinylamine vinylalcohol—molar ratio 2:1, polyvinylaminevinylformamide—molar ratio 1:2 and polyvinylamine vinylformamide-molar ratio 2:1;
Triethylenetetramine, diethylenetriamine, tetraethylenepentamine;
Bis-aminopropylpiperazine;
Polyamino acid (L-lysine/lauric acid in a molar ratio of 10/1), Polyamino acid (L-lysine/aminocaproic acid/adipic acid in a molar ratio of 5/5/1), ), Polyamino acid (L-lysine/aminocaproic acid/ethylhexanoic acid in a molar ratio of 5/3/1) Polyamino acid (polylysine-cocaprolactam); Polylysine hydrobromide; cross-linked polylysine,
amino substituted polyvinylalcohol with a MW ranging from 400-300,000;
polyoxyethylene bis[amine] available from e.g. Sigma;
polyoxyethylene bis[6-aminohexyl] available from e.g. Sigma;
N,N′-bis-(3-aminopropyl)-1,3-propanediamine linear or branched (TPTA); and
1,4-bis-(3-aminopropyl)piperazine (BNPP).

Preferred amino functional polymers containing at least one primary and/or secondary amine group are:
polyvinylamines with a MW ranging from 600, 1200, 3K, 20K, 25K or 50K;
amino substituted polyvinylalcohol with a MW ranging from 400-300,000;
polyoxyethylene bis[amine] available from e.g. Sigma;
polyoxyethylene bis[6-aminohexyl] available from e.g. Sigma;
N,N′-bis-(3-aminopropyl)-1,3-propanediamine linear or branched (TPTA);
1,4-bis-(3-aminopropyl)piperazine (BNPP);
cross-linked polylysine,
Polylysine hydrobromide.

Furthermore, such amino functional polymers comprising at least one primary and/or secondary amine group and the amine reaction product provide fabric appearance benefits, in particular color care and protection against fabric wear. Indeed, the appearance of fabrics, e.g., clothing, bedding, and household fabrics like table linens is one of the area of concern to consumers. Indeed, upon typical consumer's uses of the fabrics such as wearing, washing, rinsing and/or tumble-drying of fabrics, a loss in the fabric appearance; which can be at least partly due to loss of color fidelity and color definition, is observed. Such a problem of color loss is even more acute after multiwash cycles. It has been found that the compositions of the present invention provide improved fabric appearance and protection against fabric wear and improved color care to laundered fabrics, especially after multiwash cycles.

Therefore, the compositions of the present invention can provide simultaneously fabric care and long lasting perfume benefits.

Additionally, the Schiff base forming amine group may be part of a molecule such as one substantive to substrates such that articles of manufacture comprising the substrate may be treated with the Schiff base to attach the Schiff base thereto. Over time, the biodegradation of the Schiff base releases the fragrant aldehydic musk, whose aroma is unadulterated by the non-volatile amine also released. The instability of the released aldehydic musk would be of no consequence in such applications since it is smelled immediately soon after release. Suitable such amines include polyamines such as polylysine or naturally occurring protamine sulfate.

One of the primary advantages associated with the invention concerns the fact that the methyl ketone musks conventionally employed in industry are less biodegradable than the corresponding aldehydes (more stable in the environment) and, therefore, tend to bioaccumulate in and prejudice the environment. The aldehydic musks derivatized according to the present invention are much more unstable in the environment since they are readily oxidized to the acid, which is water-soluble or hydrophilic and, therefore, less likely to be accumulated. Moreover, the aldehydes are expected to be more easily degraded by bacteria etc than the methylketones (see, e.g., Biowin and MITI EPA—http://www.epa.gov/opptintr/exposure/docs/episuite.htm).

Therefore, the aldehydic musks released by the derivatives of the invention do not tend to bioaccumulate in the environment.

Novel fragrance and flavor aromachemicals are disclosed herein as well as methods of making the derivatives, uses of the aromachemicals and articles of manufacture including the aromachemicals. These novel derivatives find utility in any and all applications requiring certain aroma themes. The invention also relates to mixtures of these derivatives, methods for their preparation and their use as perfume materials for application to a variety of substrates.

Examples of suitable articles of manufacture in which the derivatives of the invention may be incorporated include perfumes and colognes, candles, air fresheners, detergent compositions and disinfectants.

The compositions, products, preparations and articles in which the compounds and derivatives of the invention may be incorporated include candles, air fresheners, perfumes, fragrances, colognes, soaps, bath or shower gels, shampoos or other hair care products, cosmetic preparations, body odorants, deodorants or antiperspirants, liquid or solid fabric detergents or softeners, bleach products (hypochlorites), disinfectants, all-purpose household or industrial cleaners, foods, flavorings, beverages such as beer and soda, denture cleansers (tablets), flavored orally-delivered products such as lozenges, candies, chewing gums, matrices, pharmaceuticals and the like.

The compounds can be used as perfuming ingredients, as single compounds or as mixture thereof, preferably at a range of at least about 30% by weight of the perfume composition, more preferably at a range of at least about 60% by weight of the composition. The compounds can even be used in their pure state or as mixtures, without added components. The olfactive characteristics of the individual compounds are also present in mixtures thereof, and mixtures of these compounds can be used as perfuming ingredients. This may be particularly advantageous where separation and/or purification steps can be avoided by using compound mixtures.

The derivatives of the invention can be included in virtually any article of manufacture that can include conventional aromachemicals, or for that matter, other fragrances, whether natural or artificial. Examples include bleach, detergents, flavorings and fragrances, beverages, including alcoholic beverages, and the like. The derivatives of the invention can be used in applications like soaps, shampoos, body deodorants and antiperspirants, solid or liquid detergents for treating textiles, fabric softeners, detergent compositions and/or all-purpose cleaners for cleaning dishes or various surfaces, for both household and industrial use. Of course, the use of the compounds is not limited to the above-mentioned products, as they be used in other current uses in perfumery, namely the perfuming of soaps and shower gels, hygiene or hair-care products, as well as of body deodorants, air fresheners and cosmetic preparations, and even in fine perfumery, namely in perfumes and colognes. The products of the invention also find utility in foods, flavorings, beverages such as beer and soda, denture cleansers (tablets), flavored orally-delivered products such as lozenges, candies, chewing gums, matrices, pharmaceuticals and the like. These uses are described in more detail below.

In all cited applications, the derivatives of the invention can be used alone, in admixture with each other, or in admixture with other perfuming ingredients, solvents or adjuvants of current use in the art. The nature and the variety of these co-ingredients do not require a more detailed description here, which, moreover, would not be exhaustive, and the person skilled in the art will be able to choose the latter through its general knowledge and as a function of the nature of the product to be perfumed and of the desired olfactive effect.

These perfuming ingredients typically belong to chemical classes as varied as alcohols, aldehydes, ketones, esters, ethers, acetates, nitrites, terpene hydrocarbons, sulfur- and nitrogen containing heterocyclic compounds, as well as essential oils of natural or synthetic origin. A large number of these ingredients described in reference textbooks such as the book of S. Arctander, Perfume and Flavor Chemicals, 1969, Montclair, N.J., USA, the contents of which are hereby incorporated by reference in its entirety, or its more recent versions, or in other works of similar nature.

The proportions in which the derivatives of the invention can be incorporated in the various products vary within a large range of values. These values depend on the nature of the article or product that one desires to perfume and the odor effect searched for, as well as on the nature of the co-ingredients in a given composition when the compounds are used in admixture with perfuming co-ingredients, solvents or adjuvants of current use in the art.

As an example, the derivatives of the invention are typically present at concentrations between about 0.1 and about 10%, or even more, by weight of these compounds relative to the weight of the perfuming composition in which they are incorporated. Far lower concentrations than those mentioned above can be used when the compounds are directly applied for perfuming the various consumer products cited beforehand.

The compounds may be used in detergents containing bleaching agents and activators such as, for example, tetraacetylethylenediamine (TAED), hypohalites, in particular hypochlorite, peroxygenated bleaching agents such as, for example, perborates, etc. The compounds can also be used in body deodorants and antiperspirants, for example, those containing aluminum salts. These embodiments are described in more detail below.

In addition to the derivatives described herein, the compositions herein include a detersive surfactant and optionally, one or more additional detergent ingredients, including materials for assisting or enhancing cleaning performance, treatment of the substrate to be cleaned, or to modify the aesthetics of the detergent composition (e.g., perfumes, colorants, dyes, etc.). Non-limiting examples of synthetic detersive surfactants useful herein typically at levels from about 0.5% to about 90%, by weight, include the conventional Cl-18 alkyl benzene sulfonates (“LAS”) and primary, branch-chain and random ClO-20 alkyl sulfates (“AS”), and the like.

Preferred compositions incorporating only synthetic detergents have a detergent level of from about 0.5% to 50%. Compositions containing soap preferably comprise from about 10% to about 90% soap.

The compositions herein can contain other ingredients such as enzymes, bleaches, fabric softening agents, dye transfer inhibitors, suds suppressors, and chelating agents, all well known within the art.

The derivatives described herein can be incorporated into beverages and impart various flavorings to the beverages. The beverage composition can be a cola beverage composition, and can also be coffee, tea, dairy beverage, fruit juice drink, orange drink, lemon-lime drink, beer, malt beverages, or other flavored beverage. The beverages can be in liquid or powdered form. The beverage compositions can also include one or more flavoring agents; artificial colorants; vitamin additives; preservatives; caffeine additives; water; acidulants; thickeners; buffering agents; emulsifiers; and or fruit juice concentrates. Artificial colorants which may be used include caramel color, yellow 6 and yellow 5. Useful vitamin additives include vitamin B2, vitamin B6, vitamin B12, vitamin C (ascorbic acid), niacin, pantothenic acid, biotin and folic acid. Suitable preservatives include sodium or potassium benzoate. Salts which may be used include sodium, potassium and magnesium chloride. Exemplary emulsifiers are gum arabic and purity gum, and a useful thickener is pectin. Suitable acidulants include citric, phosphoric and malic acid, and potential buffering agents include sodium and potassium citrate.

In one embodiment, the beverage is a carbonated cola beverage. The pH is generally about 2.8 and the following ingredients can be used to make the syrup for these compositions: Flavor Concentrate, including one or more of the derivatives described herein (22.22 ml), 80% Phosphoric Acid (5.55 g), Citric Acid (0.267 g), Caffeine (1.24 g), artificial sweetener, sugar or corn syrup (to taste, depending on the actual sweetener) and Potassium Citrate (4.07 g). The beverage composition can be prepared, for example, by mixing the foregoing syrup with carbonated water in a proportion of 50 ml syrup to 250 ml of carbonated water. Flavored food and pharmaceutical compositions including one or more of the derivatives described herein can also be prepared. The derivatives can be incorporated into conventional foodstuffs using techniques well known to those of skill in the art. Alternatively, the derivatives can be incorporated within polymeric particles, which can, in turn, be dispersed within and/or over a surface of an orally-deliverable matrix material, which is usually a solid or semi-solid substrate. When used in chewable compositions, the derivatives can be released into the orally-deliverable polymeric matrix material as the composition is chewed and held in the mouth, thus prolonging the flavor of the composition. In the case of dried powders and mixes, the flavor can be made available as the product is consumed or be released into the matrix material as the composition is further processed. When two flavors are combined with the polymeric particles, the relative amounts of the additives can be selected to provide simultaneous release and exhaustion of the compounds.

In one embodiment, the flavored composition includes an orally-deliverable matrix material; a plurality of water insoluble polymeric particles dispersed in the orally-deliverable matrix material, where the polymeric particles individually define networks of internal pores and are non-degradable in the digestive tract; and one or more derivatives as described herein entrapped within the internal pore networks. The derivatives are released as the matrix is chewed, dissolved in the mouth, or undergoes further processing selected from the group consisting of liquid addition, dry blending, stirring, mixing, heating, baking, and cooking. The orally-deliverable matrix material can be selected from the group consisting of gums, latex materials, crystallized sugars, amorphous sugars, fondants, nougats, jams, jellies, pastes, powders, dry blends, dehydrated food mixes, baked goods, batters, doughs, tablets, and lozenges.

A flavorless gum base can be combined with a derivative of the invention or other suitable derivative as described herein to a desired flavor concentration. Typically, a blade mixer is heated to about 11 OF, the gum base is preheated so that it is softened, and the gum base is then added to the mixer and allowed to mix for approximately 30 seconds. The flavored derivative is then added to the mixer and mixed for a suitable amount of time. The gum can be then removed from the mixer and rolled to stick thickness on waxed paper while warm.

In one embodiment, the derivatives described herein are incorporated into a system which can release a fragrance in a controlled manner. These include substrates such as air fresheners, laundry detergents, fabric softeners, deodorants, lotions, and other household items. The fragrances are generally one or more derivatives of essential oils as described herein, each present in different quantities U.S. Pat. No. 4,587,129, the contents of which are hereby incorporated by reference in their entirety, describes a method for preparing gel articles which contain up to 90% by weight of fragrance or perfume oils. The gels are prepared from a polymer having a hydroxy (lower alkoxy) 2-alkeneoate, a hydroxy (lower alkoxy) lower alkyl 2-alkeneoate, or a hydroxy poly (lower alkoxy)lower alkyl 2-alkeneoate and a polyethylenically unsaturated crosslinking agent. These materials have continuous slow release properties, i.e., they release the fragrance component continuously over a long period of time. Advantageously, all or a portion of those derivatives that include an aldehyde group can be modified to include an acetal group, which can cause the formulations to release fragrance over a period of time as the acetal hydrolyzes to form the aldehyde compound.

The formation of the Schiff base compounds of the invention proceeds according to the following reaction scheme:

R₁—CHO+H₂N—R₂→R1-CHOH—NH—R₂→R1CH═N—R₂+H₂O

wherein R₁ is the residue of the aldehydic musk and R₂ is the residue of the Schiff base forming amine.

### Example

A mixture of the amine and musk aldehyde is stirred in a suitable solvent (e.g., ethanol, dipropylene glycol, diisopropyl myristate) until imine formation is complete as judged by thin layer chromatography or NMR. Additives such as acids (e.g., paratoluene sulfonic acid) and dehydrating agents (e.g., molecular sieves/sodium sulfate/magnesium sulfate) may be used to accelerate the reaction. Elevated temperatures can be employed also to improve the condensation. When complete the reaction is worked up in an appropriate manner (e.g., filtering to remove insoluble additives/washing to remove additives) and concentrated to yield the product.

I claim:

1. A compound comprising a Schiff's base of an aldehydic musk aromachemical with a substantially nonvolatile, odorless amine, wherein the imine moiety of said Schiff's base compound is stable against oxidation to a carboxyl group or a mixture of said compounds; said Schiff's base compound being biodegradable over time to said aldehydic musk and said nonvolatile, odorless amine.

2. A compound of claim 1 wherein said aldehydic musk is a benzenic musk having the structure: 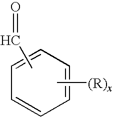
wherein: R is a straight or branched chain, saturated or unsaturated hydrocarbyl group; preferably, alkyl or alkenyl, having 1-8 carbon atoms, and
x is 1-5.

3. The compound of claim 1 wherein said aldehydic musk is a polycyclic musk having the structure: 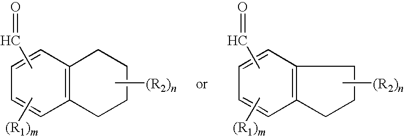
wherein: R₁ and R₂ may be the same or different and are R is a straight or branched chain, saturated or unsaturated hydrocarbyl group; preferably, alkyl or alkenyl, having 1-8 carbon atoms, and
m is 1-4 and n is 1-6.

4. The compound of claim 1 wherein said amine is an odorless, low vapor pressure aliphatic or aromatic amine containing at least one free, unmodified primary and/or secondary amino.

5. The compound of claim 4 wherein said amine is aminobenzenesulfonic acid or anthranilic acid.

6. The compound of claim 1 wherein said aldehydic musk is 2,4-ditertiarybutyl-5-methoxybenzaldehyde.

7. The compound of claim 1 wherein said aldehydic musk has the formula:

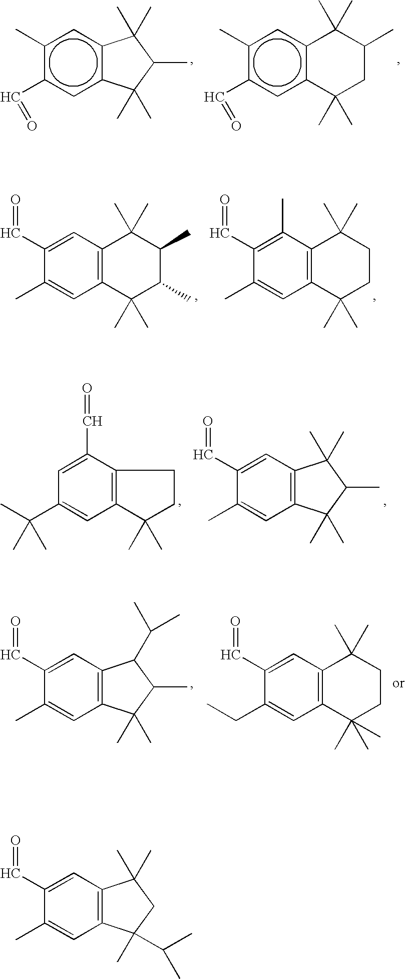

8. A composition, product, preparation or article having improved aroma, fragrance or odor releasing characteristics containing as active ingredient a compound or mixture of compounds of claim 1.

9. The composition, product, preparation or article of claim 8, wherein the compound or mixture of compounds is present in an amount of at least 30 percent by weight.

10. The composition, product, preparation or article of claim 8, wherein the compound or mixture of compounds is present in an amount of at least 60 percent by weight.

11. A composition, product, preparation or article of claim 8 in the form of a perfume, fragrance or cologne, a soap, a bath or shower gel, a shampoo or other hair care product, a cosmetic preparation, a body odorant, deodorant or antiperspirant, an air freshener, a liquid or solid fabric detergent or softener, bleach product, disinfectant or an all-purpose household or industrial cleaner.

12. A perfuming composition, product, preparation or article of claim 11, wherein the compound or mixture of compounds is present in admixture with other perfuming ingredients, solvents, or adjuvants of current use in the art.

13. A perfumed article according to claim 11, in the form of a perfume or cologne, a soap, a bath or shower gel, a shampoo or other hair care product, a cosmetic preparation, a body deodorant or antiperspirant, an air freshener, a fabric detergent or softener or an all-purpose household cleaner.

14. A composition, product, preparation or article of claim 13 in the form of a perfume wherein the compound or mixture of compounds is in admixture with other perfuming ingredients, solvents or adjuvants.

15. A body deodorant or antiperspirant composition, product, preparation or article, of claim 13.

16. A body deodorant or antiperspirant composition, product, preparation or article, of claim 15, wherein the compound or mixture of compounds is present in admixture with other perfuming ingredients, solvents, or adjuvants of current use in the art.

17. A detergent composition, product, preparation or article of claim 13.

18. A detergent composition, product, preparation or article of claim 17 wherein the compound or mixture of compounds is in admixture with other detergent ingredients, solvents or adjuvants.

19. A bleach composition, product, preparation or article of claim 13.

20. A bleach composition, product, preparation or article of claim 19 wherein the compound or mixture of compounds is in admixture with other bleach ingredients, solvents or adjuvants.

21. A composition, product, preparation or article of claim 13 in the form of a disinfectant.

22. The disinfectant composition, product, preparation or article of claim 21 wherein the compound or mixture of compounds is in admixture with other disinfectant ingredients, solvents or adjuvants.

23. A composition, product, preparation or article having improved flavor or taste characteristics containing as active ingredient a compound or mixture of compounds of claim 1.

24. A composition, product, preparation or article of claim 23 in the form of a beverage.

25. A beverage composition, product, preparation or article of claim 24 wherein the compound or mixture of compounds is in admixture with other beverage ingredients, solvents or adjuvants.

26. A composition, product, preparation or article of claim 23 in the form of a flavoring.

27. A flavoring composition, product, preparation or article of claim 26 wherein the compound or mixture of compounds is in admixture with other flavoring ingredients, solvents or adjuvants.

28. A composition, product, preparation or article of claim 23 in the form of a food.

29. A food composition, product, preparation or article of claim 28 wherein the compound or mixture of compounds is in admixture with other food ingredients, solvents or adjuvants.

30. A composition, product, preparation or article of claim 23 in the form of a chewing gum.

31. A chewing gum composition, product, preparation or article of claim 30 wherein the compound or mixture of compounds is in admixture with other chewing gum ingredients, solvents or adjuvants.

32. A composition, product, preparation or article of claim 8 in the form of a pharmaceutical.

33. A pharmaceutical composition, product, preparation or article of claim 32 wherein the compound or mixture of compounds is in admixture with other pharmaceutical ingredients, solvents or adjuvants.

34. A composition, product, preparation or article of claim 23 in the form of an orally-deliverable matrix material.

35. A composition, product, preparation or article of claim 34 wherein the compound or mixture of compounds is in admixture with other matrix material ingredients, solvents or adjuvants.

36. A method to confer, improve, enhance or modify the taste or flavor property of a composition, product, preparation or article which comprises adding thereto a flavor effective amount of a compound or mixture of compounds of claim 1.

37. The method of claim 36 wherein said composition, product, preparation or article is in the form of a beverage.

38. The method of claim 36 wherein said composition, product, preparation or article is in the form of a flavoring.

39. The method of claim 36 wherein said composition, product, preparation or article is in the form of a food.

40. The method of claim 36 wherein said composition, product, preparation or article is in the form of a chewing gum.

41. The method of claim 36 wherein said composition, product, preparation or article is in the form of a pharmaceutical.

42. The method of claim 36 wherein said composition, product, preparation or article is in the form of an orally deliverable matrix.

43. A method to confer, improve, enhance or modify the aroma, fragrance or odor characteristics of a composition, product, preparation or article which comprises adding thereto an aroma, fragrance or odor effective amount of a compound or mixture of compounds of claim 1.

44. The method of claim 43 wherein said composition, product, preparation or article is in the form of a perfume.

45. The method of claim 43 wherein said composition, product, preparation or article is in the form of a body odorant, deodorant or antiperspirant.

46. The method of claim 43 wherein said composition, product, preparation or article is in the form of a detergent.

47. The method of claim 43 wherein said composition, product, preparation or article is in the form of a bleach product.

48. The method of claim 43 wherein said composition, product, preparation or article is in the form of a disinfectant.

49. An article of manufacture comprising packaging material and an aroma, odor, fragrance, taste or flavor enhancing agent contained within said packaging material, wherein said agent is effective for the enhancement of the aroma, odor, fragrance, taste or flavor of a composition, preparation, product or article to which it is added, and wherein said packaging material comprises a label which indicates that said agent can be used for enhancing aroma, odor, fragrance, taste or flavor, and wherein said agent is a compound or mixture of compounds of claim 1.

50. A method of stabilizing an aldehydic musk aromachemical compound against oxidation of its aldehydic group to a carboxyl group comprising forming a Schiff's base thereof with a substantially nonvolatile and odorless amine whereby said aldehydic group is converted to an imine moiety.

51. The method of claim 50 wherein said aldehydic musk is a benzenic musk having the structure: 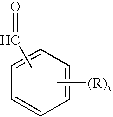
wherein: R is a straight or branched chain, saturated or unsaturated hydrocarbyl group; preferably, alkyl or alkenyl, having 1-8 carbon atoms, and
x is 1-5.

52. The method of claim 50 wherein said musk is 2,4-ditertiarybutyl-5-methoxybenzaldehyde.

53. The method of claim 50 wherein said aldehydic musk is a polycyclic musk.

54. The method of claim 53 wherein said polycyclic musk has the structure: 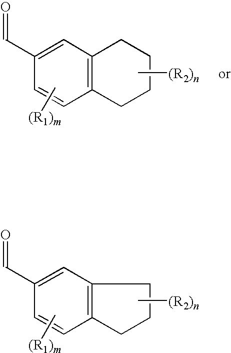
wherein: R₁ and R₂ may be the same or different and are R is a straight or branched chain, saturated or unsaturated hydrocarbyl group; preferably, alkyl or alkenyl, having 1-8 carbon atoms, and
m is 1-4 and n is 1-6.

55. The method of claim 50 wherein said amine is an odorless, low vapor pressure aliphatic or aromatic amines containing at least one free, unmodified primary and/or secondary amino.

56. The method of claim 55 wherein said amine is aminobenzenesulfonic acid or anthranilic acid.

57. The method of claim 50 wherein said aldehydic musk has the formula:

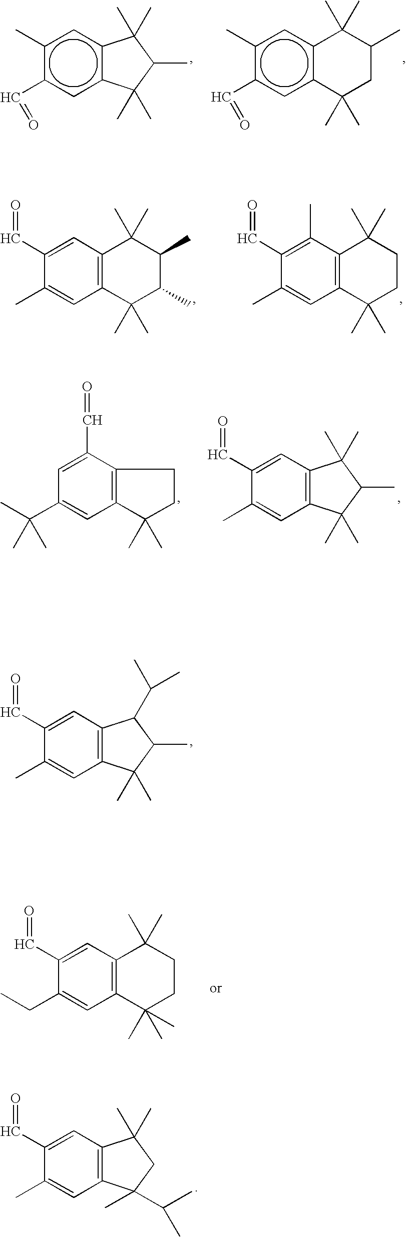

58. A benzenic or polycyclic aldehydic musk aromachemical, excluding 2,4-ditertiarybutyl-5-methoxybenzaldehyde.

59. A benzenic aldehydic musk aromachemical of claim 58 having the formula: 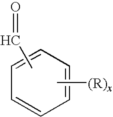
wherein: R is a straight or branched chain, saturated or unsaturated hydrocarbyl group; preferably, alkyl or alkenyl, having 1-8 carbon atoms, and
x is 1-5.

60. A polycyclic aldehydic musk of claim 58 having the formula: 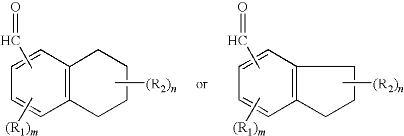
wherein: R₁ and R₂ may be the same or different and are R is a straight or branched chain, saturated or unsaturated hydrocarbyl group; preferably, alkyl or alkenyl, having 1-8 carbon atoms, and
m is 1-4 and n is 1-6.

61. A benzenic or polycyclic aldehydic musk aromachemical of claim 58 having the formula: [chemical structure]
